(19) United States
(12) Patent Application Publication
(10) Pub. No.: US 2006/0240082 A1
(43) Pub. Date: Oct. 26, 2006

(54) Method for manufacture of improved cleaning and washing products

(76) Inventor: Pan Hua Kui, Gvangxi (CN)

Correspondence Address: LAW OFFICE OF MORRIS E. COHEN, 1122 CONEY ISLAND AVENUE, SUITE 217, BROOKLYN, NY, 11230, US

(21) Appl. No.: 11/345,618

(22) Filed: Jan. 31, 2006

Related U.S. Application Data
(60) Provisional application No. 60/648,651, filed on Jan. 31, 2005.

(51) Int. Cl. A61K 36/47; A61K 9/70; A61K 9/14

(52) U.S. Cl. 424/443; 424/489; 424/731

(57) ABSTRACT

A method for creation of an improved cleaning product having an antimicrobial composition integrated therein.

### RELATED APPLICATIONS

The present application claims the priority of U.S. Provisional Application Ser. No. 60/648,651 filed Jan. 31, 2005, which is fully incorporated herein by reference.

### BACKGROUND OF THE INVENTION

Numerous products, such as poufs, sponges, and the like, are currently utilized by consumers for cleaning and/or washing in the home and office, and in related contexts such as in automobiles and so forth. Such products come into contact with large numbers of microorganisms which are deposited onto the product during use. Accordingly, it would be advantageous to provide an improved method for manufacture of a product designed to prevent spread or growth of those microorganisms.

### SUMMARY OF THE INVENTION

The present invention relates to a new method for manufacture of a cleaning or washing product, with the product preferably being an article of manufacture such as a pouf, whether a bath pouf for body use, or one designed for use in any other context where washing of a person or article may be desired.

Further to the invention, a pouf is manufactured which includes an antimicrobial composition that has been integrated into the pouf's material. In this manner, the composition inhibits the growth of and/or kills undesired microorganisms. As a result, microbial growth is inhibited when the pouf is stored and not in use. Likewise, use of the pouf inhibits growth of or kills microorganisms on surfaces that the pouf comes into contact with.

Further objects and advantages of the invention will become apparent in conjunction with the detailed disclosure provided herein.

### BRIEF DESCRIPTION OF THE FIGURES

FIG. 1 is a rear view of a bath pouf having an elastic handle attached thereto, in accordance with one embodiment of the present invention.

FIG. 2 is a front view of a bath pouf attached to a mitt into which a user can slip his or her hand, wherein the pouf surrounds the mitt, in accordance with a further embodiment of the present invention.

FIG. 3 is a rear view of a bath pouf attached to a mitt into which a user can slip his or her hand, wherein the pouf is provided only on the bottom of the mitt, in accordance with a further embodiment of the present invention.

FIG. 4 is a rear view of a bath pouf having a hard rubber grip handle attached thereto, in accordance with a further embodiment of the present invention.

### DETAILED DESCRIPTION OF THE INVENTION AND PREFERRED EMBODIMENTS

The present invention relates to a new washing device, such as a bath pouf, a pouf for washing automobiles, or so forth. Alternatively, the invention can be used as a new form of sponge or other washing material.

In accordance with the invention, the pouf includes an antimicrobial composition used to inhibit the growth of and/or kill microorganisms. As a result, the pouf (or other article) resists mold, mildew and other microbial growth.

Further in accordance with the invention, the composition is not merely a coating or chemical treatment on the surface of the pouf, but rather, is integrated into the material of the pouf itself. Since the composition is integrated into the articles' material, it does not rub off or wear off, as with a coating. Rather, as the material wears down, antimicrobial composition is still presented on the material's surface.

Accordingly, in a first step of the invention, a suitable plastic material is selected and placed into a blender for mixing. In a preferred embodiment of the invention, PE is utilized. Preferably, the material is LDPE. Further preferably, the material is provided in bead form (i.e. particular form, such particles being of any shape, whether spheres or so forth). Further preferably, small particles are utilized, or, if the material is in large form it-is preferably ground into smaller particles for ease of use.

In a second step of the invention, an oil is provided. Preferably, castor oil is used, or another suitable oil which is preferably non-toxic. The oil is placed in the blender and mixed with the plastic beads, with the blender being activated for a suitable period so as to coat each bead's surface. For example, the blender may be run for 2-3 minutes, or for such period required so as evenly distribute the oil over the surface of all of the beads.

In a third step of the invention, a suitable antimicrobial composition is selected. In one preferred embodiment, the antimicrobial composition is triclosan. Alternatively, another suitable composition can be utilized wherein the composition provides the desired characteristics, such as antibacterial, antiviral and/or antifungal properties.

A desired amount of the antimicrobial composition is then mixed into the blender to adhere the antimicrobial composition to the oil coating the surface of each of the beads. The antimicrobial composition used can be in particulate or liquid form. For example, triclosan in powdered form can be provided, so as to distribute the powder over the beads, and stick that powdered composition to the oil.

In an alternate embodiment, the antimicrobial composition is mixed into the oil prior to application of the oil to the beads. Further alternatively, although an oil is preferred, the antimicrobial composition can be dissolved in any other suitable substance, whether in a liquid in a viscous state, or so forth, for application of that substance to the beads. Or, the antimicrobial can be applied directly to the bead's surface if it is in a state which will adhere directly to the bead (e.g., in its pure liquid form, or as a powdered solid).

In a preferred embodiment wherein the plastic is LDPE, the oil is castor oil, and the antimicrobial composition is triclosan, the materials are preferably mixed using relative weights of 96% LDPE, 2% castor oil, and 2% triclosan. Alternatively, any other suitable mixture or ratio providing a desired amount of antimicrobial composition can be utilized.

Once the plastic, the oil, and the antimicrobial composition have been mixed to provide a homogeneous composition, this composition is then placed into a plastic injection machine to form a plastic mesh. As a result, in accordance with the method of the invention, a plastic mesh is formed which has been impregnated with the antimicrobial composition of interest so as to form a homogeneous distribution of the antimicrobial composition throughout the plastic.

In the next step, as with traditional pouf manufacture, the mesh then is placed through a water pool to cool down, and is packed into rolls for storage until desired use, or for use immediately, as applicable. Any of the suitable methods known in the art can be used to make the pouf. Generally, the worker weighs the mesh and provides a length of the desired size and weight, with the mesh being cut to that desired length. The mesh is then folded to the correct size, and a rope is used to tie the mesh in a ball shape, or the desired shape for the final pouf. Examples of methods for manufacture of poufs are further provided in U.S. Pat. Nos. 5,766,700; 5,946,780; and 6,443,527; all of which are fully incorporated herein by reference.

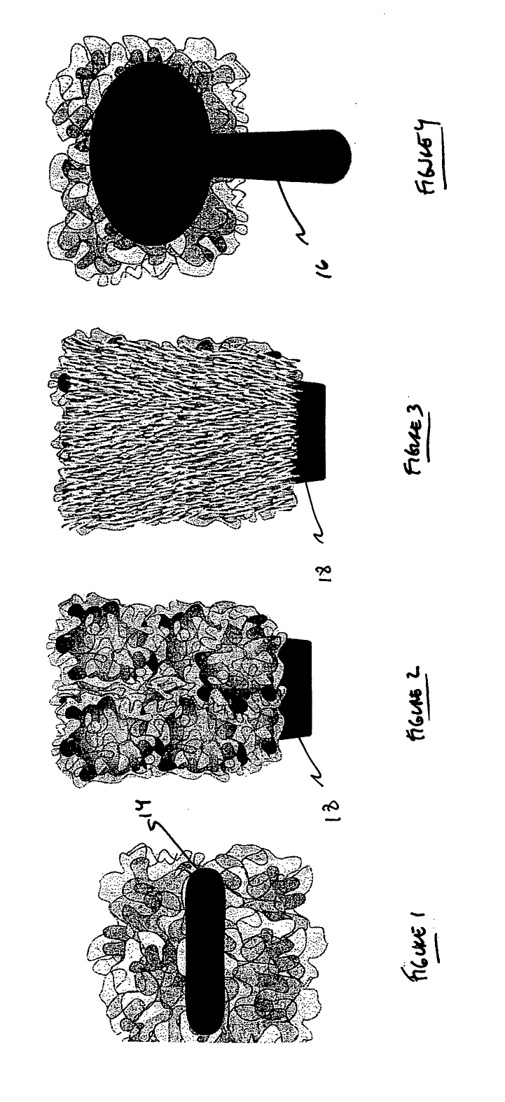

In addition, in further embodiments of the invention, the pouf can include a handle as shown, for example, in FIGS. 1 and 4. For example, the mesh of the pouf can be wound around or glued to the handle, to assist with manipulation of the pouf during use. This handle can be elastic or some other flexible material, as with handle 14 of FIG. 1; or, a plastic or hard rubber handle 16 can be utilized, as shown in FIG. 4.

In yet further embodiments, the mesh of the pouf can be attached to a mitt 18 for scrubbing (or can be tightly woven to form a mitt), as shown in FIGS. 2 and 3. The provision of a mitt configuration allows a user to place his or her hand within the mitt, also to ease manipulation of the pouf during use. Likewise, although a bath pouf is a preferred embodiment, in further embodiments of the invention, the device can be any other form of pouf. For example, consistent with the invention, other forms of poufs are intended as well, such as poufs for automotive care, for household use, for hospital care, or so forth. Further in accordance with the invention, the impregnated material can be provided in any other article of manufacture used for washing and/or cleaning so as to provide antimicrobial properties.

Having described this invention with regard to specific embodiments, it is to be understood that the description is not meant as a limitation since further embodiments, modifications and variations may be apparent or may suggest themselves to those skilled in the art. It is intended that the present application cover all such embodiments, modifications and variations.

What is claimed is:

1. A method, comprising the steps of:
(a) taking beads comprising plastic;
(b) coating said beads with oil;
(c) mixing an antimicrobial composition into said oil;
(d) forming a plastic mesh from said beads coated with said oil and coated with said antimicrobial composition; and,
(e) forming a pouf from said plastic mesh.

2. A method as claimed in claim 1, wherein said antimicrobial composition is mixed into said oil after said beads are coated with said oil.

3. A method as claimed in claim 1, wherein said antimicrobial composition is mixed into said oil before said beads are coated with said oil.

4. A method as claimed in claim 1, wherein said pouf is provided with a handle.

5. A method as claimed in claim 1, wherein said plastic is LDPE.

6. A method as claimed in claim 1, wherein said antimicrobial composition is triclosan.

7. A method as claimed in claim 1, wherein said pouf further comprises a mitt for scrubbing.

8. A method as claimed in claim 1, wherein said materials are provided in relative weights of 96% of said plastic to 2% of said oil, to 2% of said antimicrobial composition.

9. A method as claimed in claim 1, wherein said plastic is LDPE, said oil is castor oil, and said antimicrobial composition is triclosan, and wherein said materials are provided in relative weights of 96% of said LDPE to 2% of said castor oil, to 2% of said triclosan.

10. A method as claimed in claim 1, wherein said pouf is provided for use in the bath.

11. A method as claimed in claim 1, wherein said pouf is provided for automotive care.

12. A method as claimed in claim 1, wherein said pouf is provided for household use,

13. A method as claimed in claim 1, wherein said pouf is provided for use in a hospital.
